(19) United States
(12) Patent Application Publication
(10) Pub. No.: US 2013/0065928 A1
(43) Pub. Date: Mar. 14, 2013

(54) BIOMARKERS FOR PI3K-DRIVEN CANCER

(71) Applicants: Anke Klippel-Giese, Park Ridge, NJ (US); Keziban Unsal-Kacmaz, Upper Saddle River, NJ (US)

(72) Inventors: Anke Klippel-Giese, Park Ridge, NJ (US); Keziban Unsal-Kacmaz, Upper Saddle River, NJ (US)

(73) Assignee: WYETH LLC, Madison, NJ (US)

(21) Appl. No.: 13/638,925

(22) PCT Filed: Apr. 2, 2011
(86) PCT No.: PCT/IB2011/051419

§ 371 Date: Nov. 30, 2012

Related U.S. Application Data
(60) Provisional application No. 61/320,963, filed on Apr. 5, 2010.
(60) Provisional application No. 61/322,071, filed on Apr. 8, 2010.

(51) Int. Cl. G01N 33/574; A61K 31/4439; A61P 35/00; A61K 31/4409

(52) U.S. Cl. 514/341; 435/7.4; 514/352

(57) ABSTRACT

Disclosed is the discovery that the mTORC2 complex plays a role in the regulation of PKN3 phosphorylation at the turn motif threonine; and the use of the phosphorylation status of the turn motif threonine of PKN3 as a biomarker. In some embodiments, the phosphorylation status of the turn motif threonine of PKN3 is determined using an antibody that specifically binds to the turn motif threonine of a PKN3 protein, such as an anti-phosphoT860 antibody. In some embodiments, the invention relates to methods for screening compounds that have cancer therapeutic potential, methods for diagnosing cancer, methods for determining the prognosis of a patient suffering from cancer, methods for stratifying patients in a clinical trial, methods for treating a patient suffering from cancer, and methods for determining the effectiveness of a particular treatment regimen.

This application is a 371 of PCT/IB2011/051419, filed Apr. 2, 2011, which claims the benefit of U.S. Provisional Application No. 61/320,963, filed Apr. 5, 2010 and U.S. Provisional Application No. 61/322,071 filed on Apr. 8, 2010, both of which are hereby incorporated by reference in their entirety.

### REFERENCE TO SEQUENCE LISTING

This application is being filed electronically via EFS-Web and includes an electronically submitted sequence listing in .txt format. The .txt file contains a sequence listing entitled “PC60620A_Sequence_Listing_ST25.txt” created on Sep. 4, 2012 and having a size of 31 KB. The sequence listing contained in this .txt file is part of the specification and is herein incorporated by reference in its entirety.

### BACKGROUND OF THE INVENTION

This application is directed to methods for selecting cancer patients for treatment of cancer or for stratification of patients in trials for cancer treatments. Specifically, the application relates to the use of phosphorylated threonine at a helix turn locus in a PKN3 protein as a biomarker for identifying or stratifying patients who may respond to a particular cancer therapy.

The development of effective cancer therapies increasingly relies on the elucidation of the molecular mechanisms underlying the disease, and the identification of target molecules within those mechanisms which may be useful in the development of new drugs. Once such target molecules are available, drug candidate compounds can be tested against those targets. In many cases, such drug candidates are members of a compound library which may consist of synthetic or natural compounds.

There is significant need to identify new molecular targets associated with particularly aggressive forms of cancer so that new therapeutic compounds and regimens can be identified and validated.

Many forms of cancer involve an aberrantly active phosphatidylinositol 3-kinase (PI3K) pathway. Aberrant PI3K pathway activity is generally thought to be caused by loss of the PTEN tumor suppressor and/or activating mutations in PI3K. Recently, Guertin et al. have shown that the mTOR complex 2 (mTORC2) coactivates Akt along with PI3K and is required for PTEN minus human prostate epithelial cells to form tumors in mice (Guertin et al., Cancer Cell 15:148-159, 2009). mTORC2 comprises a serine/threonine protein kinase FK506 binding protein-12-rapamycin associated protein 1 (a.k.a. mammalian target of rapamycin; mTOR), mLST8/GβL, Rictor, SIN1 and PROTOR/PRR5.

Thus, the elucidation of upstream and downstream components of the mTORC2 pathway will enhance the discovery and deployment of agents that impinge upon mTORC2 activity for the treatment of particular forms of cancer involving the PI3K pathway.

### SUMMARY

The inventors have made the surprising discovery that mTORC2 participates in the activation of PKN3 by phosphorylating the turn motif threonine of PKN3, which is usually assigned the position of T860.

In one aspect, the invention provides a method of treating a patient suffering from cancer, which includes the steps of (a) obtaining a tumor sample from the patient, (b) determining the level of phosphorylation of a turn motif threonine of a PKN3 protein in the tumor sample (“test level”), (c) comparing the test level to a reference level of phosphorylation of a turn motif threonine of a PKN3 protein (“reference level”), and (d) administering a cancer therapeutic compound to the patient, wherein the compound decreases mTORC2 pathway activity in a cell. Based on the results of the comparison step, the patient is selected to receive the cancer treatment.

In a second aspect, the invention provides a method for selecting a patient that is capable of responding to a cancer therapeutic agent, wherein the agent decreases mTorC2 pathway activity in a cell, comprising the steps of (a) obtaining a tumor sample from the patient, (b) determining the level of phosphorylation of a turn motif threonine of a PKN3 protein in the tumor sample (“test level”), (c) comparing the test level to a reference level of phosphorylation of a turn motif threonine of a PKN3 protein (“reference level”), and (d) selecting the patient for treatment with the cancer therapeutic agent. Based on the results of the comparison, which can be displayed to an end-user in a graphic or written form, the practitioner determines whether the patient is capable of responding to the cancer treatment and selects the patient based on that determination.

In a third aspect, the invention provides a method for determining the effectiveness of a compound in the treatment of cancer in a patient, comprising the steps of (a) administering a cancer therapeutic compound to the patient, wherein the compound decreases mTorC2 pathway activity in a cell, (b) obtaining a test tumor sample from the patient at a time after the administering step (“test sample”), (c) determining the level of phosphorylation of a turn motif threonine of a PKN3 protein in the test sample (“test level”) and (d) comparing the test level to a reference level phosphorylation of a turn motif threonine of a PKN3 protein (“reference level”). Based on the results of the comparison, which may be displayed to an end user, the practitioner determines whether the compound has had any effect on the amelioration of the cancer in the patient.

In one embodiment of the three aforementioned aspects, the reference level and test level of phosphorylation of the turn motif are each determined using an antibody that specifically binds to the turn motif threonine of a PKN3 protein. In some embodiments, the PKN3 protein has a sequence similar or identical to SEQ ID NO:1, of which the turn motif threonine is the threonine at residue number 860 (“T860”). In some embodiments, the antibody that specifically binds to the turn motif threonine of a PKN3 protein is an anti-phosphoT860 antibody. The antibody may be a polyclonal or monoclonal antibody.

In some embodiments of the aforementioned aspects, the reference level of phosphorylation of the turn motif threonine is the level of phosphorylation of the turn motif threonine of a PKN3 protein found in non-cancerous tissue of the patient, or an average level found in non-cancerous tissues from several patients, donors or tissue types. In other embodiments, the reference level is the level found in a particularly aggressive form of cancer known to involve mTORC2 activity, or an average of levels in cancers from several sources. In still other embodiments, the reference level is an arbitrary level, which in some embodiments is based upon clinical responses of patients to a given drug, or upon ex vivo cell responses, or upon responses of particular patient groups.

In some embodiments of the third aspect, the reference level of phosphorylation of the turn motif threonine is the level of phosphorylation of the turn motif threonine of a PKN3 protein found in a tumor sample obtained from the patient prior to administration of the cancer therapeutic compound.

In a fourth aspect, the invention provides for the use of an anti-phosphoT860 antibody in the selection of a patient capable of responding to a cancer therapeutic compound that decreases mTorC2 pathway activity in a cell, wherein the anti-phosophoT860 antibody binds to a phosphorylated turn motif threonine of a PKN3 protein.

In some embodiments of the fourth aspect, anti-phosphoT860 antibody is a polyclonal antibody. In other embodiments, the anti-phosphoT860 antibody is a monoclonal antibody.

In some embodiments of the fourth aspect, the patient is selected for participation in a clinical trial to determine the safety and/or efficacy of a cancer therapeutic compound that decreases mTORC2 pathway activity in a cell.

In some embodiments of any of the aforementioned aspects, the mTORC2 pathway activity is the phosphorylation of the turn motif threonine of a PKN3 protein. In other embodiments, the mTorC2 pathway activity is the activation of a Rho GTPase. In still other embodiments, the mTorC2 pathway activity is the phosphorylation of Akt.

In some embodiments of any of the aforementioned aspects, the cancer therapeutic compound is targeted against a cancer that is PI3K-driven, which includes prostate cancer.

### DRAWINGS

FIG. 1 depicts a Western blot showing doxycycline-induced expression of wild-type and kinase-dead PKN3, phosphorylated PKN3 (at the turn motif threonine) and phosphorylated substrate (GSKα).

FIG. 2 depicts a Western blot showing the effects of changing concentrations of Y27632, SB202190 and SB202474 on the expression of PKN3, phosphorylated PKN3 (at the turn motif threonine) and phosphorylated substrate (GSKα).

FIG. 3 depicts a Western blot showing the effects of changing concentrations of Y27632 on the expression of PKN3, phosphorylated PKN3 (at the turn motif threonine) and phosphorylated PKN1 and PKN2.

FIG. 4 depicts a Western blot showing the effects of changing concentrations of the kinase inhibitors staurosporin, WAY-125132 and CCI-779 in the presence or absence of Y27632 on the expression of phosphorylated kinase-dead PKN3-T860, phosphorylated PKN3-T718, phosphorylated AKT and phosphorylated S6K.

FIG. 5 depicts a Western blot showing the effects of changing concentrations of the kinase inhibitors staurosporin, WAY-125132 and CCI-779 in the presence or absence of Y27632 on the expression of phosphorylated wild-type PKN3-T860, phospho-PKN3-T718, phosphorylated AKT and phosphorylated S6K.

FIG. 6 depicts photomicrographs of HEK293T cells transfected with wild-type (panels A, B and C) or kinase-dead (panels D, E and F) PKN3 constructs under control of a doxycycline responsive promoter, in the absence of doxycycline (panels A and D), in the presence of doxycycline (panels B and E) or in the presence of doxycycline and WAY-125132 (panels C and F).

FIG. 7 depicts a Western blot showing the effects of Raptor antisense expression, Rictor antisense expression or mTor antisense expression on the expression of phospho-PKN3-T860 and phospho-AKT-S473 in cells that express wild-type PKN3, kinase-dead PKN3 or kinase-dead PKN3 in the presence of Y27632.

### DETAILED DESCRIPTION

It is generally known that the catalytic activity of PKN3 requires phosphorylation events within its kinase domain at two conserved sites, namely at a threonine in its activation loop (e.g., “T718”), which is likely to be phosphorylated by PDK1, and at a threonine in its turn motif (e.g., “T860”), which is phosphorylated by a heretofore unknown upstream kinase. In an effort to elucidate the unknown kinase responsible for phosphorylating the turn motif threonine of PKN3, applicants generated an activation-state specific antibody against the turn-motif phosphorylation site at threonine 860 (T860) of human PKN3, and used that antibody to help to ascertain the mechanism by which PKN3 is activated. This antibody was used to probe the status of PKN3 in doxycycline responsive cell lines.

The applicants have made the surprising discovery that phosphorylation of PKN3 at both sites is not dependent on the intrinsic kinase activity of PKN3, but rather on an active conformation of the nucleotide binding pocket of PKN3. It was discovered that a kinase inactive mutant of PKN3 is not phosphorylated at these sites, unless its ATP-binding pocket is occupied by an ATP-competitive inhibitor of PKN3. Furthermore, by probing this property of the kinase-inactive enzyme in combination with the T860 antibody, the applicants made the surprising discovery that the mammalian target of rapamycin complex 2 (“mTORC2”) is required for phosphorylation of PKN3 at the turn-motif site (T860), and that this phosphorylation event is likely required for its function in tumorigenesis.

Accordingly, the applicants envision use of the phosphorylation-state-specific T860 antibody as an important biomarker tool for patient stratification and monitoring therapeutic response. The applicants further envision the use of the above described assay system, which allows kinase-defective PKN3 (“PKN3kd”) variants to adopt an active catalytic center conformation combined with the phosphor-T860-antibody, as a robust cell-based screening regimen for identifying mTORC2-specific inhibitors, which have cancer-therapeutic potential.

PKN3 is a serine/threonine protein kinase of 889 amino acid residues in length (human orthologue). It has an N-terminal putative regulatory region containing three antiparallel coiled-coil (ACC) domains ACC1, ACC2 and ACC3 located at about residues 15-77, 97-170 and 184-236, respectively; a C-terminal catalytic region located at residues 559-882; and a C2-like domain of about 100 to 130 residues in length positioned between the putative regulatory domain and the catalytic domain. There are at least three different isoforms of PKN (PKN1/PKNα/PAK-1/PRK-1, PKN2/PRK2/PAK-2/PKNγ, and PKN3/PKNβ) in mammals, each of which shows different enzymological properties, tissue distribution, and varied functions. For a review of PKN, see Mukai, H., J. Biochem. 133:17-27, 2003. See also U.S. Patent Application No: 20040106569, published Jun. 3, 2004, which is incorporated herein by reference in its entirety.

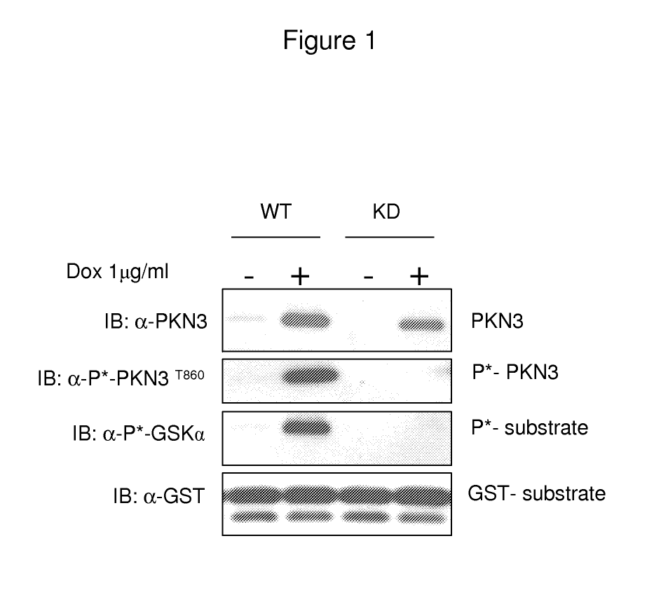

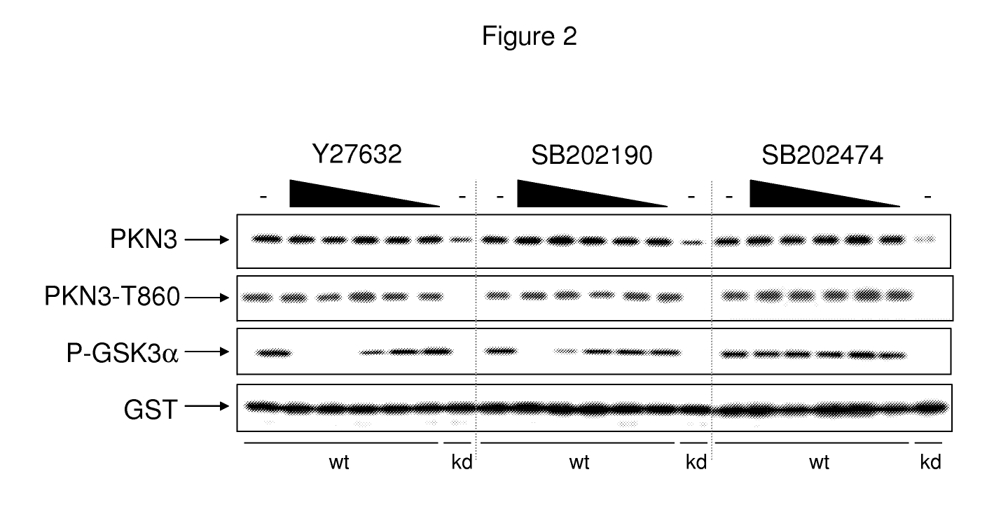

Applicants have previously shown that PKN3 is up-regulated in cancer cells having increased aggressiveness and drug resistance (see FIGS. 1 and 2, respectively of copending U.S. Provisional Application Nos. 61/159,739 and 61/226,078, which are incorporated herein by reference in their entirety). By increased aggressiveness, what is meant is that the cancer cells are metastatic, have high potential to metastasize, have increased rate of proliferation, or are drug resistant. An aggressive cancer is exemplified by, e.g., a triple-negative breast cancer (see, e.g., Dent et al., Clinical Cancer Research 13: 4429-4434, Aug. 1, 2007). Aggressive cancers also comprise those cancers in which the mTORC2/PKN3/RhoC pathway is involved.

Compounds that inhibit the activity of mTORC2 and/or PKN3 (or other effectors in the PKN3 pathway of activity) can be used to control metastatic and proliferational behavior of cells and therefore provide methods of treating tumors and cancers, more particularly those tumors and cancers which are aggressive. The reduction in signaling and other activities that are effected by mTORC2 and/or PKN3 activity may stem either from a reduction at the transcription level, at the level of the translation, or at the level of post-translational modification (e.g., phosphorylation activation of PKN3) of one or more of the mTORC2/PKN3 pathway components, or at the level of quaternary structure formation (i.e., formation of a ternary complex involving PKN3).

Because of the involvement of mTORC2 in the activation of PKN3, especially in the etiology of aggressive cancer, PKN3 that is phosphorylated at the turn motif threonine (e.g., T860) can be used as a prognostic marker, a disease staging marker, a patient-stratification marker, or a marker for diagnosing the status of a cell or patient having in his body such kind of cells as to whether the patient is capable of responding to a cancer therapeutic compound that targets mTORC2 activity.

PKN3 is a developmentally regulated mediator of PI3K-induced migration and invasion of cells. It is regulated by PI3K at the level of expression and catalytic activity in an Akt-independent manner. It has a restricted expression pattern (endothelial, embryonic and tumor cells) and is not essential for most normal cell function. It is required for metastatic PC-3 (PTEN−/−) cell growth in an orthotopic mouse model.

In normal cells, the PI3-kinase (phosphatidyl-inositol-3-kinase) pathway is characterized by a PI3-kinase activity upon growth factor induction and a parallel signaling pathway. Growth factor stimulation of cells leads to activation of their cognate receptors at the cell membrane which in turn associate with and activate intracellular signaling molecules such as PI3-kinase. Activation of PI3-kinase (consisting of a regulatory p85 and a catalytic p110 subunit) results in activation of Akt by phosphorylation, thereby supporting cellular responses such as proliferation, survival or migration further downstream. PTEN is thus a tumor suppressor which is involved in the phosphatidylinositol (PI) 3-kinase pathway and which has been extensively studied in the past for its role in regulating cell growth and transformation (for reviews, see, e.g., Stein, R. C. and Waterfield, M. D. Mol Med Today 6:347-357, 2000).

The tumor suppressor PTEN functions as a negative regulator of PI3-kinase by reversing the PI3-kinase-catalyzed reaction and thereby ensures that activation of the pathway occurs in a transient and controlled manner. Chronic hyperactivation of PI3-kinase signaling is caused by functional inactivation of PTEN. PI3-kinase activity can be blocked by addition of the small molecule inhibitor LY294002. The activity and downstream responses of the signaling kinase MEK which acts in a parallel pathway, can, for example, be inhibited by the small molecule inhibitor PD98059.

Chronic activation of the PI3-kinase pathway through loss of PTEN function is a major contributor to tumorigenesis and metastasis, indicating that this tumor suppressor represents an important checkpoint for a controlled cell proliferation. PTEN knock-out cells show similar characteristics as those cells in which the PI3-kinase pathway has been chronically induced via activated forms of PI3-kinase. Activation of phosphatidylinositol 3-kinase is sufficient for cell cycle entry and promotes cellular changes characteristic of oncogenic transformation.

The mammalian target of rapamycin (mTOR) is a serine/threonine protein kinase that regulates cell growth, cell proliferation, cell motility, cell survival, protein synthesis, and transcription. mTOR Complex 2 (mTORC2) comprises mTOR, rapamycin-insensitive companion of mTOR (Rictor), GβL, and mammalian stress-activated protein kinase interacting protein 1 (mSIN1). mTORC2 has been shown to phosphorylate the serine/threonine protein kinase Akt/PKB at a serine residue S473. Phosphorylation of the serine stimulates Akt phosphorylation at a threonine T308 residue by PDK1 and leads to the full activation of Akt. mTORC2 is known to be important to the development of PTEN-related cancers (see Facchinetti et al., EMBO J. 2008 Jul. 23; 27(14):1932-43; and Guertin et al., Cancer Cell. 2009 Feb. 3; 15(2):148-59, which are incorporated herein by reference).

Diseases and conditions involving dysregulation of the PI3-kinase pathway are well known. Any of these conditions and diseases may thus be addressed by the inventive methods and the drugs and diagnostic agents, the design, screening or manufacture thereof is taught herein. For reasons of illustration but not limitation conditions and diseases are referred to the following: endometrial cancer, colorectal carcinomas, gliomas, endometrial cancers, adenocarcinomas, endometrial hyperplasias, Cowden's syndrome, hereditary non-polyposis colorectal carcinoma, Li-Fraumene's syndrome, breast cancer, ovarian cancer, prostate cancer, Bannayan-Zonana syndrome, LDD (Lhermitte-Duklos' syndrome), hamartoma-macrocephaly diseases including Cow disease (CD) and Bannayan-Ruvalcaba-Rily syndrome (BRR), mucocutaneous lesions (e.g., trichilemmonmas), macrocephaly, mental retardation, gastrointestinal harmatomas, lipomas, thyroid adenomas, fibrocystic disease of the breast, cerebellar dysplastic gangliocytoma and breast and thyroid malignancies.

In view of this, activated phosphorylated PKN3 and its associated effectors (e.g., mTORC2 and RhoC) are valuable drug targets downstream of the PI3-kinase pathway which can be addressed by drugs which will have less side effects than other drugs directed to upstream targets. Thus, the present invention provides a drug target which is suitable for the design, screening, development and manufacture of pharmaceutically active compounds which are more selective than those known in the art, such as, for example, 2-(4-morpholinyl)8-phenylchromone (“LY 294002”), which generally target PI3-kinase, and rapamycin and 2-[1-(2,4-Dichlorophenyl)-2-(1H-imidazol-1-yl)ethylidene]hydrazinecarboximidamide dihydrochloride (“WAY-125132”), which generally target mTOR (both complex 1 and 2). By having control over this particular piece of the PKN3 signaling machinery (i.e., phosphorylation at turn motif threonine) and any further downstream molecule involved in the pathway, only a very limited number of parallel branches thereof or further upstream targets in the signaling cascade are likely to cause unwanted effects. Therefore, the other activities of the PI-3 kinase/PTEN pathway related to cell cycle, DNA repair, apoptosis, glucose transport, translation will not be influenced.

The complete sequence of a nucleic acid encoding PKN3 (PKN3 is shown as SEQ ID NO:1), which is also known as protein kinase N beta (PKNβ), is generally available in public databanks (see e.g., in GENBANK accession nos: NM_{—}013355, BA85625, XM_{—}001159776, inter alia.) Also, the amino acid sequence of PKN3 is available in databanks under the accession number NP_{—}037487.2. The skilled artisan will readily recognize or expect that other PKN3 orthologs and homologs, which contain a turn motif threonine, are useful in the practice of this invention. The complete sequence of a nucleic acid encoding mTOR (mTOR is exemplified in SEQ ID NO:2) (human ortholog) is generally available in public databanks (see e.g., in GENBANK accession nos: NM_{—}004958, BC117166, L34075, inter alia.) Also, the amino acid sequence of mTOR is available in databanks under the accession numbers P42345, P42346, Q9JLN9, NP_{—}063971, NP_{—}004949 and NP_{—}064393, inter alia. The skilled artisan will readily recognize or expect that other mTOR orthologs and homologs are useful in the practice of this invention. mTOR is discussed exempli gratia in Menon, S. and Manning, B. D., Common corruption of the mTOR signaling network in human tumors, Oncogene 2008 December; 27 Suppl 2:S43-51. It is within the present invention that derivatives or truncated versions of PKN3 and mTOR and its complex 2-associated proteins may be used according to the present invention as long as the desired effects may be realized. The extent of derivatization and truncation can thus be determined by one skilled in the art by routine analysis.

In the context of the present invention, the term nucleic acid sequences encoding PKN3, mTOR, and mTORC2-associated proteins (id est mLST8/GβL, Rictor, SIN1 and PROTOR/PRR5) also include nucleic acids which hybridize to nucleic acid sequences specified by the aforementioned accession numbers or any nucleic acid sequence which may be derived from the aforementioned amino acid sequences. Such hybridization is known to the skilled artisan. The particularities of such hybridization may be taken from Sambrook, J. Fritsch, E. F. and Maniatis, T. (1989) Molecular Cloning: A Laboratory Manual, 2nd ed. Cold Spring Harbor: Cold Spring Harbor Laboratory. In a preferred embodiment, the hybridization is a hybridization under stringent conditions, for example, under the stringent conditions specified in Sambrook supra.

In addition, nucleic acids encoding a PKN3, mTOR and mTORC2-associated protein are also nucleic acid sequences which contain sequences homologous to any of the aforementioned nucleic acid sequences, whereby the degree of sequence homology is 75, 80, 85, 90 or 95%.

Orthologues to human PKN3 may be found, among others, in organisms as evolutionarily diverse as M. musculus and R norvegicus, A. thaliana, C. elegans, D. melanogaster and S. cerevisiae. In the case of PKN3, the percent identity is 67%, 51%, 38%, 36%, 63% and 44%, respectively, for the various species mentioned before. Orthologues to human mTOR are found in rodents, birds, bony fish and insects, with percent identities of 98%, 96%, 90% and 62%, respectively. It will be acknowledged by the skilled artisan that any of these or other orthologues and homologues will in principle be suitable for the practice of the present invention, provided the drug or diagnostic agent generated using such homologue may still interact with the human PKN3 or mTORC2 or any other intended PKN3 or mTORC2.

The phosphorylation status of the turn motif threonine of a PKN3 (“Phospho-PKN3 marker”), or other read-out of mTORC2 activity (“mTORC2 readout”), may be used as a biomarker for patient stratification or response of a tumor in a patient to an anti-cancer compound that targets mTOR activity, more preferably mTORC2 activity. Suitable anti-cancer compounds belonging to different classes of compounds such as antibodies, peptides, anticalines, aptamers, spiegelmers, ribozymes, antisense oligonucleotides and siRNA, as well as small organic molecules, may be used. The anti-cancer compounds may be designed, selected, screened, generated or manufactured by either using a Phospho-PKN3-based screen, or other mTORC2 readout screen. In such screening method, a first step is to provide one or several so-called candidate or test compounds. Candidate compounds as used herein are compounds the suitability of which is to be tested in a test system for treating or alleviating cancer as described herein or to be used as a diagnostic means or agent for cancer.

If a candidate compound shows a respective effect in a test system, said candidate compound is a suitable means or agent for the treatment of said diseases and disease conditions and, in principle, as well a suitable diagnostic agent for said diseases and disease conditions. In a second step, the candidate compound is contacted with a system comprising a PKN3 protein (or a fragment thereof containing a turn motif threonine) and mTORC2 (“PKN3/mTORC2 system”). The PKN3/mTORC2 system is also referred to herein as a system detecting the kinase activity of the activated phosphorylated PKN3. In some embodiments, in addition to the direct assessment of the phosphorylation state of the turn motif threonine of PKN3, the kinase activity of the activated phosphorylated PKN3 can be assessed by determining the phosphorylation of a substrate, such as, e.g., a diagnostic GSK3-derived fragment having a sequence of GPGRRGRRRTSSFAEGG (SEQ ID NO:3).

The Phospho-PKN3-based or other mTORC2 readout screening methodology described herein also is useful to eliminate non-functional or inactive compounds from further consideration. Thus, PKN3 kinase activity or phosphorylation status (generally “PKN3 status”) can be measured in a first sample obtained from a subject or test system, generating a pre-treatment level, followed by administering a test compound to the subject or test system and measuring the PKN3 status in a second sample from the subject or test system at a time following administration of the test compound, thereby generating data for a test level. The pre-treatment level (first level) can be compared to the test level (second level), and data showing no decrease in the test level relative to the pre-treatment level indicates that the test compound is not effective in the subject, and the test agent may be eliminated from further evaluation or study.

The mTORC2 readout screening methodology described herein (e.g., Phospho-PKN3-based screen) is useful to evaluate whether a patient is capable of responding to a particular anti-cancer compound, which has as its mechanism of action the interference of the phosphorylation of the turn motif threonine of PKN3. Said evaluation is useful in the stratification of patient populations for treatment purposes as well as selection of participants in clinical trials. A tumor sample is obtained from the patient and the relative amount (e.g., specific activity) of turn motif threonine phosphorylated PKN3 (e.g., P*T860) is determined. The relative amount of turn motif threonine phosphorylated PKN3 can be determined by directly measuring the level of phosphothreonine PKN3, such as with an anti-phosphothreonine antibody, or by measuring the kinase activity of the phosphothreonine PKN3, such as by measuring the activity of a PKN3 kinase substrate. Those patients showing elevated levels of phosphorylated turn motif threonine PKN3 are selected as patients who are likely to respond to a therapy targeted against mTORC2.

The mTORC2 readout screening methodology described herein (e.g., Phospho-PKN3-based screen) is also useful to evaluate whether a patient is responding or has responded to a particular anti-cancer compound, which has as its mechanism of action the interference of the phosphorylation of the turn motif threonine of PKN3. A tumor sample is obtained from the patient prior to treatment and the relative amount (e.g., specific activity) of turn motif threonine phosphorylated PKN3 (e.g., P*T860) is determined. The relative amount of turn motif threonine phosphorylated PKN3 can be determined by directly measuring the level of phosphothreonine PKN3, such as with an anti-phosphothreonine antibody, or by measuring the kinase activity of the phosphothreonine PKN3, such as by measuring the activity of a PKN3 kinase substrate. This level establishes the baseline level for a particular patient. At one or more periods of time after the initiation of treatment, a tumor sample is obtained from the patient and the level of phosphorylated turn motif threonine PKN3 (“treatment level”) is determined and compared to the initial baseline level. A decrease in the treatment level relative to the baseline level indicates that the anti-cancer therapy is efficacious.

Methods to determine the level of phosphorylated turn motif threonine PKN3 as mentioned above include detection using appropriate antibodies. A suitable antibody includes an anti-phosphoT860 antibody, which can be a polyclonal, monoclonal, or recombinant monoclonal antibody. Antibodies may be generated as known to the skilled artisan and described, e.g., by Harlow, E., and Lane, D., “Antibodies: A Laboratory Manual,” Cold Spring Harbor Laboratory, Cold Spring Harbor, N.Y., (1988). Suitable antibodies may also be generated by other well known methods, for example, by phage display selection from libraries of antibodies.

In the case of an mTORC2/phosphorylated turn motif threonine PKN3 complex, an increase or decrease of the activity of the complex may be determined in a functional kinase assay. A tumor sample or cell line derived from a tumor sample can be contacted with an anti-cancer compound and a change in the activity of the mTORC2/PKN3 system is determined. In some cases, the anti-cancer compound may be in a library of compounds, which includes inter alia libraries composed of small molecules, peptides, proteins, antibodies, or functional nucleic acids. The latter compounds may be generated as known to the skilled artisan.

The manufacture of an antibody, which is specific for the phosphorylated turn motif threonine of PKN3, is known to the skilled artisan. The antibodies of the invention include nanobodies, polyclonal antibodies, monoclonal antibodies, chimeric antibodies (e.g., humanized antibodies), and anti-idiotypic antibodies. Polyclonal antibodies are heterogeneous populations of antibody molecules derived from the sera of animals immunized with an antigen. Monoclonal antibodies are a substantially homogeneous population of antibodies that bind to specific antigens. In general, antibodies can be made, for example, using traditional hybridoma techniques (Kohler and Milstein (1975) Nature, 256: 495-499), recombinant DNA methods (U.S. Pat. No. 4,816,567), or phage display using antibody libraries (Clackson et al. (1991) Nature, 352: 624-628; Marks et al. (1991) J. Mol. Biol., 222: 581-597). For additional antibody production techniques, see Antibodies: A Laboratory Manual, eds. Harlow and Lane, Cold Spring Harbor Laboratory, 1988. The present invention is not limited to any particular source, method of production, or other special characteristics of an antibody.

The term “antibody” is also meant to include both intact molecules as well as fragments such as Fab, single chain Fv antibodies (ScFv) and small modular immunopharmaceuticals (SMIPs), which are capable of binding antigen. Fab fragments lack the Fc fragment of intact antibody, clear more rapidly from the circulation, and may have less non-specific tissue binding than an intact antibody (Wahl et al., 1983, J. Nucl. Med. 24:316-325). Chimeric antibodies are molecules, different portions of which are derived from different animal species, such as those having variable region (VH, VL) derived from, e.g., a murine monoclonal antibody and a human immunoglobulin constant region (CH1-CH2-CH3, CL). Chimeric antibodies and methods for their production are known in the art (Cabilly et al., 1984, Proc. Natl. Acad. Sci. USA 81:3273-3277; Morrison et al., 1984, Proc. Natl. Acad. Sci. USA 81:6851-6855; Boulianne et al., 1984, Nature 312:643-646; Cabilly et al., European Patent Application 125023 (published Nov. 14, 1984); Taniguchi et al., European Patent Application 171496 (published Feb. 19, 1985); Morrison et al., European Patent Application 173494 (published Mar. 5, 1986); Neuberger et al., PCT Application WO 86/01533 (published Mar. 13, 1986); Kudo et al., European Patent Application 184187 (published Jun. 11, 1986); Morrison et al., European Patent Application 173494 (published Mar. 5, 1986); Sahagan et al., 1986, J. Immunol. 137:1066-1074; Robinson et al., PCT/US86/02269 (published May 7, 1987); Liu et al., 1987, Proc. Natl. Acad. Sci. USA 84:3439-3443; Sun et al., 1987, Proc. Natl. Acad. Sci. USA 84:214-218; Better et al., 1988, Science 240:1041-1043). SMIPs are single-chain polypeptides comprising one binding domain, one hinge domain and one effector domain. SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

The antibodies which may be used according to the present invention may have one or several markers or labels. Such markers or labels may be useful to detect the antibody either in its diagnostic application or its therapeutic application. Preferably the markers and labels are selected from the group comprising avidin, streptavidin, biotin, gold and fluorescein and used, e.g., in ELISA methods. These and further markers as well as methods are, e.g., described in Harlow and Lane, supra.

In one embodiment, the antibody comprises a PKN3 activation-state-specific antibody, which recognizes the phospho-threonine at position 860 in the turn motif of PKN3 (boxed) (SEQ ID NO: 4: 847-YFEGEFTGLPPAL [symbol] TPPAPHSLLTARQQA-874). Said antibody is useful inter alia as a probe for increased PKN3 expression and activation, and as a biomarker for patient stratification and therapeutic response.

A further class of medicaments, compounds that disrupt the mTORC2/PKN3 complex, as well as diagnostic agents which may be generated using the mTORC2/PKN3 complex or components and fragments thereof, or the nucleic acid encoding said mTORC2/PKN3 complex or components and fragments thereof, are peptides which bind thereto. Such peptides may be generated by using methods according to the state of the art such as phage display. Basically, a library of peptides is generated and displayed on the surface of phage, and the displayed library is contacted with the target, in the present case, for example, the PPRC complex or components thereof. Those peptides binding to the target are subsequently removed, preferably as a complex with the target molecule, from the respective reaction. It is known to the skilled artisan that the binding characteristics, at least to a certain extent, depend on the particular experimental set-up such as the salt concentration and the like. After separating those peptides binding to the target molecule with a higher affinity or a bigger force, from the non-binding members of the library, and optionally also after removal of the target molecule from the complex of target molecule and peptide, the respective peptide(s) may subsequently be characterized.

Prior to the characterization step, an amplification step optionally may be performed such as, e.g., by propagating the peptide coding phages. In some embodiments, the characterization comprises the sequencing of the target binding peptides. Basically, the peptides are not limited in their lengths; however, peptides having a length from about 8 to 20 amino acids are generally obtained in the respective methods. The size of the libraries may be about 10² to 10¹⁸ or 10⁸ to 10¹⁵ different peptides, however, the size of the library is not limited thereto.

According to the present invention, the mTORC2/PKN3 complex or components thereof, as well as the nucleic acids encoding said mTORC2/PKN3 complex or components thereof, may be used as the target for the manufacture or development of a medicament for the treatment of an aggressive cancer, as well as for the manufacture or development of means for the diagnosis of said aggressive cancer in a screening process, whereby in the screening process small molecules or libraries of small molecules are used. This screening comprises the step of contacting the target mTORC2/PKN3 complex or components thereof (target) with a single small molecule or a variety (such as a library) of small molecules at the same time or subsequently, preferably those from the library as specified above, and identifying those small molecules or members of the library which bind to the target and disrupt the function or integrity of the mTORC2/PKN3 complex which, if screened in connection with other small molecules may be separated from the non-binding or non-interacting small molecules.

The binding and non-binding may strongly be influenced by the particular experimental set-up. In modifying the stringency of the reaction parameters, it is possible to vary the degree of binding and non-binding which allows a fine tuning of this screening process. In some embodiments, after the identification of one or several small molecules which specifically interact with the target, this small molecule may be further characterized. This further characterization may, for example, reside in the identification of the small molecule and determination of its molecular structure and further physical, chemical, biological or medical characteristics. In some embodiments, the natural compounds have a molecular weight of about 100 to 1000 Da. In some embodiments, small molecules are those which comply with Lepinski's Rule of Five, which is known to the skilled artisan (see Lipinski et al., Adv. Drug. Del. Rev., 23: 3-25, 1997). Alternatively, small molecules may also be defined such that they are synthetic-small-molecules arising from combinatorial chemistry, in contrast to natural products. However, it is to be noted that these definitions are only subsidiary to the general understanding of the respective terms in the art. Like all kinases, the PKN3 component of the mTORC2/PKN3 complex contains an ATP-binding site and drugs that are known to bind to such sites are therefore suitable candidate compounds for inhibiting PPRC function. Examples of suitable compounds include, but are not limited to, LY-27632, Ro-3 1-8220, and HA 1077, all of which are available from Calbiochem (La Jolla, Calif.).

The invention is further exemplified by the following examples, which are not limiting of the scope of the invention.

### EXAMPLE 1

### PKN3 Protein Constructs

The full-length cDNA of human PKN3 (WT or wt) was amplified by PCR and cloned into a GST-fusion expression vector under the control in a doxycycline (Dox)-inducible promoter. A kinase dead (KD or kd) version of PKN3, which comprises a K588R substitution, was also cloned into the same vector using the same strategy. The proteins were expressed in HEK293T cells transfected with the PKN3 WT and KD constructs. FIG. 1 demonstrates that production of WT and KD PKN3 is responsive to doxycycline induction. WT PKN3 is phosphorylated at the turn motif threonine (P*-PKN3^{T860}) and phosphorylates the GSKα substrate, whereas the KD version does neither (FIG. 1).

For protein extraction, cells were washed twice with cold phosphate-buffered saline (PBS) and lysed at 4° C. in lysis buffer containing 20 mM Tris (pH 7.5), 137 mM NaCl, 15% (vol/vol) glycerol, 1% (vol/vol) Nonidet P-40 (NP-40), 2 mM phenylmethylsulfonyl fluoride, 10 mg of aprotinin per ml, 20 mM leupeptin, 2 mM benzamidine, 1 mM sodium vanadate, 25 mM β-glycerolphosphate, 50 mM NaF, and 10 mM Na-pyrophosphate. Lysates were cleared by centrifugation at 14,000×g for 5 min, and aliquots of the lysates were analyzed for protein expression and enzyme activity (see below). Samples were separated by sodium dodecyl sulfate-polyacrylamide gel electrophoresis (SDS-PAGE) and transferred to nitrocellulose filters (Schleicher & Schuell). Filters were blocked in TBST buffer (10 mM Tris-HCl [pH 7.5], 150 mM NaCl, 0.05% [vol/vol] Tween 20, 0.5% [wt/vol] sodium azide) containing 5% (wt/vol) dried milk. The respective antibodies were added in TBST at appropriate dilutions. Bound antibody was detected with anti-mouse-, anti-goat, or anti-rabbit-conjugated alkaline phosphatase (Santa Cruz Biotechnology) in TBST, washed, and developed with nitroblue tetrazolium and 5-bromo-4-chloro-3-indolylphosphate (Promega). Alternatively, horseradish peroxidase-conjugated secondary antibodies were used and developed by enhanced chemiluminescence (Amersham).

PKN antibodies have been described in Leenders, 2004. PDK1, phospho-GSKα, GST, PNK3-T718, S6K-ST389 and AKT-S473 antibodies are commercially available from Cell Signaling Technology, Inc. (Beverly, Mass.). Anti-phospho-PKN3 T860 rabbit monoclonal antibodies were produced according to standard procedures (see Spieker-Polet, 1995, Proc. Natl. Acad. Sci. USA, 92:9348-9352).

### EXAMPLE 2

### Use of ATP-Competitive Inhibitors to Prime Kinase Inactive PKN3

Various ATP-type kinase inhibitors were assessed for their ability to inhibit the kinase activity of both recombinant wildtype (WT) and kinase dead (KD) versions of PKN3. Cells transfected with WT or KD versions of PKN3 were treated with known ATP-type kinase inhibitors: Y27632, SB202190 and SB202474 (an inactive form of SB202190) (Ishizaki et al., Mol. Pharmacol., 57: 976-983, 2000; Manthey et al., Journal of Leukocyte Biology, 64 (3): 409-417, 1998). Both Y27632 and SB202190, but not SB202474, were shown to inhibit the kinase activity of kinase active phosphorylated PKN3 in a concentration dependent manner using a phospho-GSKα read-out (FIG. 2). Kinase dead PKN3 was not phosphorylated at the turn motif threonine and did not phosphorylate the GSK3-derived substrate (FIG. 2).

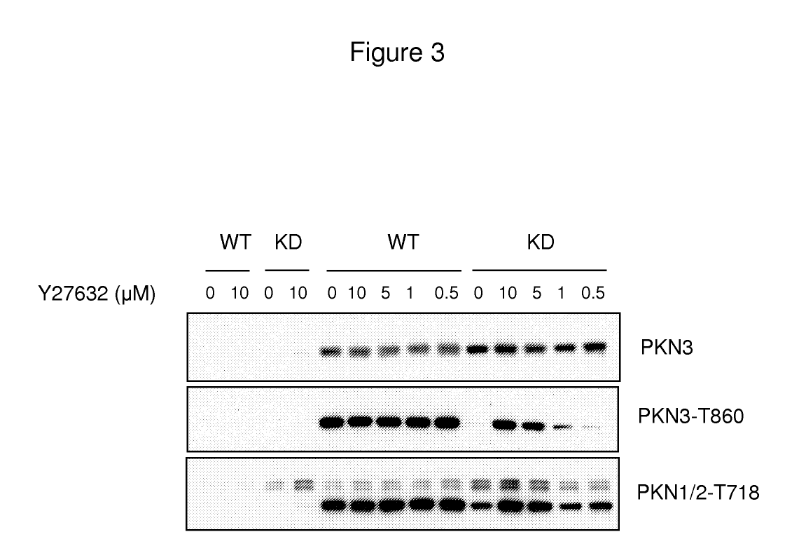

To determine if priming of PKN3 does not require intrinsic kinase activity, but rather depends on conformational regulation through the ATP binding pocket, KD PKN3 and WT PKN3 were treated with the ATP binding pocket competitive inhibitors Y27632, SB202190 and SB202474 (see Cameron et al., Nature Structural & Molecular Biology, 16(6): 624-630, 2009). Surprisingly, it was observed that both Y27632 and SB202190, but not SB202474 primed kinase dead PKN3 to become phosphorylated at the turn motif threonine in a concentration dependent manner (FIG. 3).

Y27632-primed PKN3 (WT and KD versions) was used for further studies to probe the mechanism of phosphorylation of PKN3.

### EXAMPLE 3

### Regulation of Turn Motif Phosphorylation

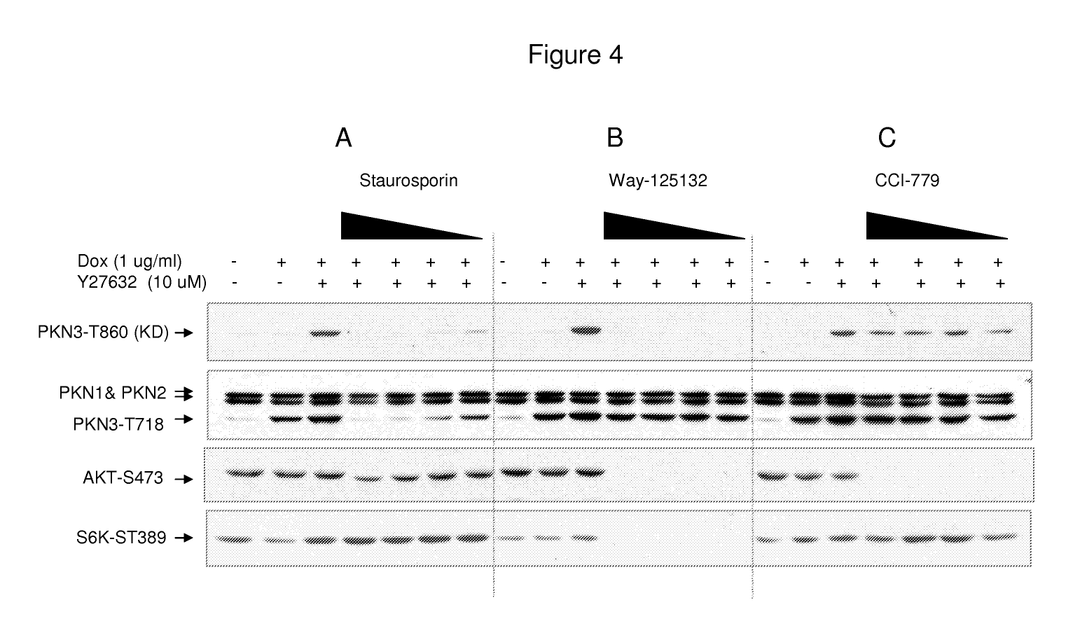

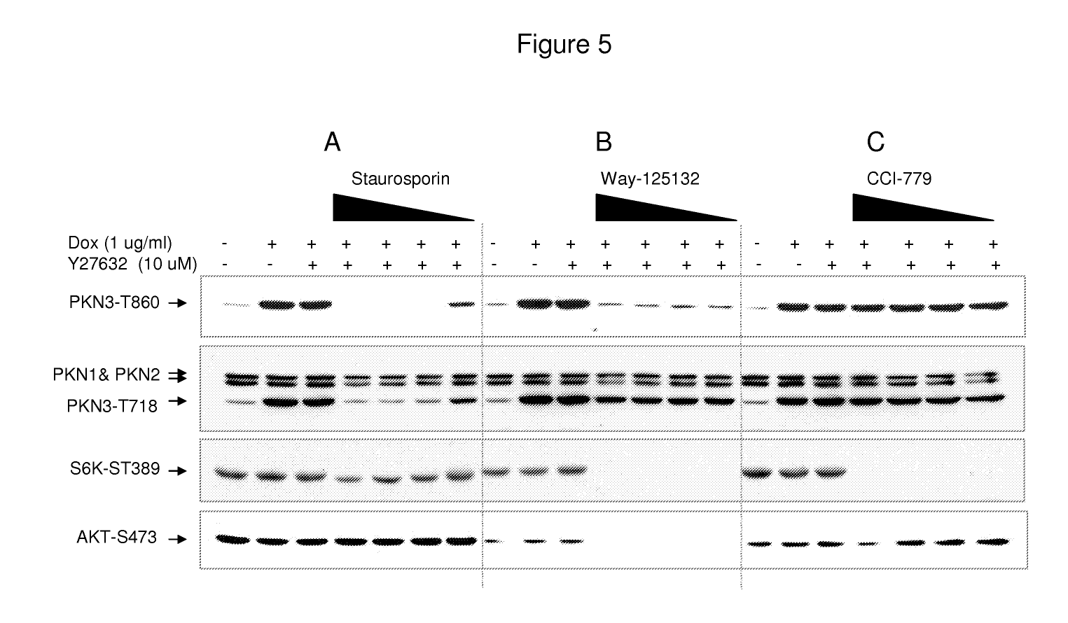

Production of both KD and WT PKN3 was induced by treating transfected cells with 1 μg/ml doxycycline for 5 hours. PKN3 was primed with 10 μM Y27632 and then treated with various kinase inhibitors for 7 hours in an effort to determine the upstream regulator of PKN3 turn motif phosphorylation (FIG. 4: KD-PKN3; FIG. 5: WT-PKN3). Staurosporin, an inhibitor of PDK1, was shown to inhibit the phosphorylation of PKN3 (WT and KD) at both the T718 and T860 sites in a concentration dependent manner (FIGS. 4 and 5, panels A). It is generally viewed in the art that PDK1 phosphorylates T718, which occurs before T860 phosphorylation. Staurosporin is believed to inhibit T860 phosphorylation by preventing T718 phosphorylation.

WAY-125132 (a.k.a. WYE-132; see WO 2009052145), a potent inhibitor of both mTORC1 and mTORC2 (see Yu et al., Cancer Research, 70(2): 621-631, Jan. 15, 2010) was shown to inhibit T860 phosphorylation in a dose dependent manner, but not T718 phosphorylation (FIGS. 4 and 5, panels B). As controls, WAY-125132 was shown to inhibit the phosphorylation of S6K-ST389, a target of mTORC1, and AKT-S473, a target of mTORC2.

CCI-779 (a.k.a. temsirolimus), an inhibitor of mTORC1 (Torneau et al., British Journal of Cancer, (2008) 99: 1197-1203). The chemical name of temsirolimus is (3S,6R,7E,9R,10R,12R,14S,15E,17E,19E,21S,23S,26R,27R,34aS)-9,10,12,13,14,21,22,23,24,25,26,27,32,33,34,34a-Hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacyclohentriacontine-1,5,11,28,29(4H,6H,31H)-pentone 4′-[2,2-bis(hydroxymethyl)propionate]; or Rapamycin, 42-[3-hydroxy-2-(hydroxymethyl)-2-methylpropanoate]. CCI-779 was shown to inhibit S6K-ST389, but had no effect on primed PKN3-T860 or PKN3-T718, PKN1, PKN2, or AKT-S473 (FIGS. 4 and 5, panels C). Taken together, these results suggest that mTORC2 has an essential function in the phosphorylation of the turn motif threonine of PKN3.

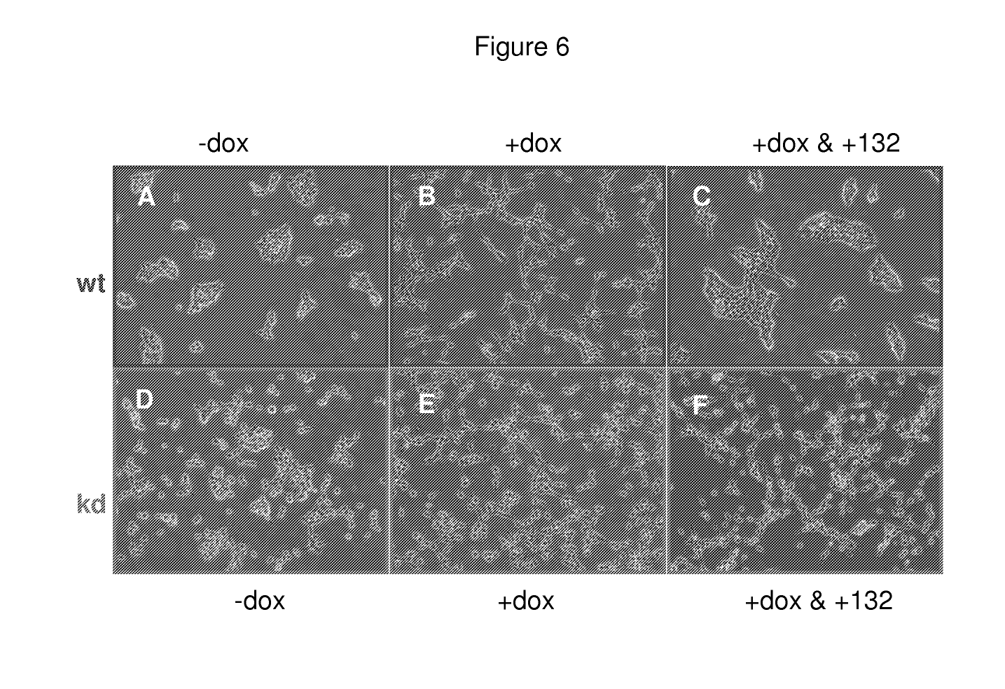

The effects of WAY-125132 on PKN3-induced morphology changes in cells were examined. Doxycycline treated cells transfected with WT PKN3 showed a transformed phenotype compared to cells not treated with doxycycline (compare FIG. 6, panel B to panel A). WAY-125132 treatment reversed this effect (FIG. 6, panel C), indicating that blocking activation of PKN3 via mTOR inhibits its cell transforming activity. KD PKN3, whether treated with WAY-125132 or not, had no effect on cell morphology (FIG. 6, panels D-F).

### EXAMPLE 4

### Requirement of mTORC2 for Phosphorylation of Turn Motif Threonine of PKN3

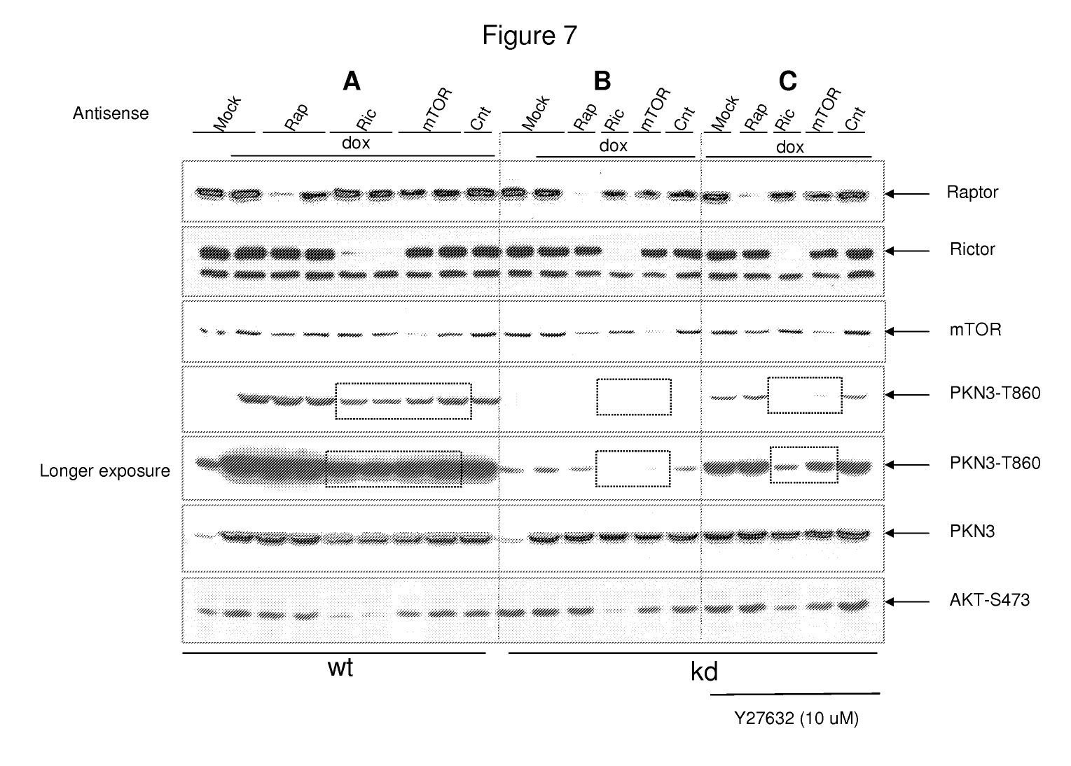

To further distinguish the role of mTORC2 versus mTORC1 in the phosphorylation of the turn motif threonine of PKN3, cells expressing either KD PKN3 or WT PKN3 were transfected with one of three antisense constructs to various mTOR complex components: raptor (a component of mTORC1), rictor (a component of mTORC2) and mTOR (a component of both). FIG. 7, panel A depicts WT PKN3 transfected with either raptor antisense (columns 3 and 4), rictor antisense (columns 5 and 6) or mTOR antisense (columns 7 and 8). FIG. 7, panel B depicts KD PKN3 transfected with either raptor antisense (column 12), rictor antisense (column 13) or mTOR antisense (column 14). FIG. 7, panel C depicts Y27632-primed KD PKN3 transfected with either raptor antisense (column 17), rictor antisense (column 18) or mTOR antisense (column 19). In every case, the raptor knockdown had no effect on the level of PKN3 turn motif phosphorylation, whereas the knockdown of either mTOR or rictor each reduced the relative amount of PKN3 phosphorylated at the turn motif threonine (e.g., PKN3-T860) (see dashed boxed regions of FIG. 7).

This result indicates that mTOR and rictor, both of which comprise mTORC2, are each required for turn motif phosphorylation of PKN3.

1. A method of treating a patient suffering from cancer, comprising:
a. obtaining a tumor sample from the patient;
b. determining a test level of phosphorylation of a turn motif threonine of a PKN3 protein in the tumor sample;
c. comparing the test level of phosphorylation of a turn motif threonine of a PKN3 protein in the tumor sample of step (b) to a reference level of phosphorylation of a turn motif threonine of a PKN3 protein; and
d. administering a cancer therapeutic compound to the patient, wherein the compound decreases mTorC2 pathway activity in a cell.

2. A method for selecting a patient that is capable of responding to a cancer therapeutic agent, wherein the agent decreases mTorC2 pathway activity in a cell, comprising:
a. obtaining a tumor sample from the patient;
b. determining a test level of phosphorylation of a turn motif threonine of a PKN3 protein in the tumor sample; and
c. comparing the test level of phosphorylation of a turn motif threonine of a PKN3 protein in the tumor sample of step (b) to a reference level of phosphorylation of a turn motif threonine of a PKN3 protein; and
d. selecting the patient when the level of step (b) is greater than the reference level.

3. A method for determining the effectiveness of a compound in the treatment of cancer in a patient, comprising:
a. administering a cancer therapeutic compound to the patient, wherein the compound decreases mTorC2 pathway activity in a cell;
b. obtaining a test tumor sample from the patient at a time after the administering step (a);
c. determining a test level of phosphorylation of a turn motif threonine of a PKN3 protein in the test tumor sample of step (d); and
d. comparing the test level of step (c) to a reference level phosphorylation of a turn motif threonine of a PKN3 protein.

4. The method of claim 1, wherein the reference level and test level of phosphorylation of the turn motif are each determined using an anti-phosphothreonine antibody specific to the turn motif threonine of a PKN3 protein.

5. The method of claim 1, wherein the turn motif threonine is T860 of SEQ ID NO:1.

6. The method of claim 4, wherein the antibody is an anti-phosphoT860 antibody.

7. The method of claim 1, wherein the reference level of phosphorylation of the turn motif threonine is the level of phosphorylation of the turn motif threonine of a PKN3 protein found in non-cancerous tissue.

8. The method of claim 1, wherein the reference level of phosphorylation of the turn motif threonine is an arbitrary value.

9. The method of claim 3, wherein the reference level of phosphorylation of the turn motif threonine is the level of phosphorylation of the turn motif threonine of a PKN3 protein found in a tumor sample obtained from the patient prior to administration of the cancer therapeutic compound.

10. The method of claim 1, wherein the mTorC2 pathway activity is the phosphorylation of the turn motif threonine of a PKN3 protein.

11. The method of claim 1, wherein the mTorC2 pathway activity is the activation of a Rho GTPase.

12. The method of claim 1, wherein the mTorC2 pathway activity is the phosphorylation of Akt.

13. The method of claim 1, wherein the cancer is a pI3K-driven cancer.

14. The method of claim 1, wherein the cancer is a prostate cancer.

15. The use of an anti-phosphoT860 antibody in the selection of a patient capable of responding to a cancer therapeutic compound that decreases mTorC2 pathway activity in a cell, wherein the anti-phosophoT860 antibody binds to a phosphorylated turn motif threonine of a PKN3 protein.

16. The use according to claim 15, wherein the anti-phosphoT860 antibody is a polyclonal antibody.

17. The use according to claim 15, wherein the anti-phosphoT860 antibody is a monoclonal antibody.

18. The use according to claim 15, wherein the patient is selected for participation in a clinical trial to determine the safety, efficacy or both of a cancer therapeutic compound that decreases mTorC2 pathway activity in a cell.

19. The use according to claim 15, wherein the cancer therapeutic compound is targeted against a cancer that is PI3K-driven.

20. The use according to claim 15, wherein the cancer therapeutic compound is targeted against prostate cancer.


SEQUENCE LISTING (4 sequences)

SEQ ID NO: 1 - protein, 889 aa, Homo sapiens
MEEGAPRQPGPSQWPPEDEKEVIRRAIQKELKIKEGVENLRRVATDRRHLGHVQQLLRSSNRRLEQLHGELRELHARILLPGPGPGPAEPVASGPRPWAEQLRARHLEALRRQLHVELKVKQGAENMTHTCASGTPKERKLLAAAQQMLRDSQLKVALLRMKISSLEASGSPEPGPELLAEELQHRLHVEAAVAEGAKNVVKLLSSRRTQDRKALAEAQAQLQESSQKLDLLRLALEQLLEQLPPAHPLRSRVTRELRAAVPGYPQPSGTPVKPTALTGTLQVRLLGCEQLLTAVPGRSPAAALASSPSEGWLRTKAKHQRGRGELASEVLAVLKVDNRVVGQTGWGQVAEQSWDQTFVIPLERARELEIGVHWRDWRQLCGVAFLRLEDFLDNACHQLSLSLVPQGLLFAQVTFCDPVIERRPRLQRQERIFSKRRGQDFLRASQMNLGMAAWGRLVMNLLPPCSSPSTISPPKGCPRTPTTLREASDPATPSNFLPKKTPLGEEMTPPPKPPRLYLPQEPTSEETPRTKRPHMEPRTRRGPSPPASPTRKPPRLQDFRCLAVLGRGHFGKVLLVQFKGTGKYYAIKALKKQEVLSRDEIESLYCEKRILEAVGCTGHPFLLSLLACFQTSSHACFVTEFVPGGDLMMQIHEDVFPEPQARFYVACVVLGLQFLHEKKIIYRDLKLDNLLLDAQGFLKIADFGLCKEGIGFGDRTSTFCGTPEFLAPEVLTQEAYTRAVDWWGLGVLLYEMLVGECPFPGDTEEEVFDCIVNMDAPYPGFLSVQGLEFIQKLLQKCPEKRLGAGEQDAEEIKVQPFFRTTNWQALLARTIQPPFVPTLCGPADLRYFEGEFTGLPPALTPPAPHSLLTARQQAAFRDFDFVSERFLEP

SEQ ID NO: 2 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 3 - protein, 17 aa, Homo sapiens
GPGRRGRRRTSSFAEGG

SEQ ID NO: 4 - protein, 28 aa, Homo sapiens
YFEGEFTGLPPALTPPAPHSLLTARQQA